(19) United States
(12) Patent Application Publication
(10) Pub. No.: US 2025/0025410 A1
(43) Pub. Date: Jan. 23, 2025

(54) COMPOSITION COMPRISING HER2 ANTIBODY, FORMULATION AND PROCESS OF PREPARATION THEREOF

(71) Applicant: ENZENE BIOSCIENCES LIMITED, Pune Maharashtra (IN)

(72) Inventors: Himanshu GADGIL, Pune Maharashtra (IN); Varda KOLAPKAR, Pune Maharashtra (IN); Nildip CHAUHAN, Pune Maharashtra (IN); Abijar BHORI, Pune Maharashtra (IN); Narahari Chittoor RAO, Pune Maharashtra (IN)

(73) Assignee: ENZENE BIOSCIENCES LIMITED, Pune Maharashtra (IN)

(21) Appl. No.: 18/714,875

(22) PCT Filed: Nov. 30, 2022
(86) PCT No.: PCT/IB2022/061582

§ 371 Date: May 30, 2024

(30) Foreign Application Priority Data
Nov. 30, 2021 (IN) 202121055313

(51) Int. Cl. A61K 9/00; A61K 9/08; A61K 47/12; A61K 47/26; C07K 16/32

(52) U.S. Cl. CPC A61K 9/0019; A61K 9/08; A61K 47/12; A61K 47/26; C07K 16/32

(57) ABSTRACT

The present invention relates to pharmaceutical composition comprising HER2 antibody, its analogues or complexes. The present invention further relates to a liquid formulation comprising HER2 antibody, its analogues or complexes and processes for preparing the same and methods of using it. More particularly, the present invention relates to a liquid formulation comprising Pertuzumab, Trastuzumab, and process for preparing the formulations.

### FIELD OF THE INVENTION

The present invention relates to pharmaceuticals. Specifically, the present invention relates to a pharmaceutical composition comprising HER2 antibody, analogues or complexes thereof. The present invention further relates to a liquid stable composition comprising HER-2 Antibody, analogues or complexes thereof, processes for preparing the composition and methods of using it. More particularly, the present invention relates to a liquid composition comprising Pertuzumab, Trastuzumab or other HER2 antibodies, analogues or complexes thereof, formulations thereof, processes for preparing and methods of using the same.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

HER2 (human epidermal growth factor receptor 2) is a gene that can play a role in the development of breast cancer. HER2, also known as ErbB2, is a member of the HER receptor family, which also consists of epidermal growth factor receptor (EGFR, ErbB1 or HER1. ErbB3 or HER3, ErbB4 or HER4) receptors. Over expression of HER2 is considered a biomarker of malignant tumors. HER2 is also over expressed in approximately 20% of gastroesophageal junction and gastric cancers with a poor prognosis.

Trastuzumab and pertuzumab are currently approved HER2 targeted antibodies. These are indicated for the treatment of patients with metastatic breast cancer. Trastuzumab is a humanized IgG1 kappa monoclonal antibody that selectively binds with high affinity to the extracellular domain of the human epidermal growth factor receptor 2 protein, HER2. Pertuzumab is a recombinant humanized monoclonal antibody that targets the extracellular dimerization domain (Subdomain II) of the human epidermal growth factor receptor 2 protein (HER2).

U.S. Pat. No. 8,372,396 discloses formulation of HER2 antibody with histidine acetate buffer, sucrose and polysorbate 20. U.S. Pat. No. 9,345,661 discloses HER2 antibody formulation comprising buffer, stabilizer and non-ionic surfactant. U.S. Pat. No. 9,345,661 specifically discloses use of first stabilizer and second stabilizer in the HER2 antibody formulation wherein α,α-trehalose dihydrate or sucrose is used as a first stabilizer and methionine as a second stabilizer. WO 2020017901 discloses HER2 antibody formulation comprising sugar, buffer and stabilizer and WO2018/004260 discloses stable liquid formulation comprising antibody, acetate or histidine buffer. EP1802344B1 and U.S. Pat. No. 9,017,671B2 discloses antibody formulations, including monoclonal antibodies formulated in histidine-acetate buffer, as well as a formulation comprising an antibody that binds to domain II of HER2 (for example, pertuzumab), and a formulation comprising an antibody that binds to death receptor 5 (DR5). U.S. Pat. No. 9,968,676B2 discloses subcutaneous anti-HER2 antibody formulations and uses thereof.

While there have been attempts as mentioned above in recent years to develop compositions and formulations comprising HER2 targeted antibodies, many have limited application because of their limited stabilities. Excipients used in formulations appears to be one of the key factors affecting the stability of the drug products like antibodies. One of the most commonly used excipients in formulations are buffers, which can contribute significantly to the overall stability of a drug product through various mechanisms Most of the formulations of the afore mentioned patent documents and those known in the art include amino acid based buffer and/or include amino acids as an antioxidant. During storage conditions, such amino acid can undergo degradation in formulation and also catalyse degradation of surfactant like polysorbate 20 and polysorbate 80. Polysorbate degradation is reported to be highest in histidine chloride buffer. Polysorbates are amphiphilic, non-ionic surfactants, and they represent one of the key components of biopharmaceuticals. Polysorbates degradation can lead to lowered surfactant protection of the drug product against interfacial stress and the unwanted impact of the polysorbate degradation products on the stability of the protein (Kishore. R. S. K., et al., Pharm Res 28. 1194-1210) (2011)). Therefore, it is crucial to use suitable buffer to prevent degradation of polysorbates. As proteins have very complex and diverse structures, it is very difficult to predict a priori the best excipients for any given protein molecule. Antibodies are considered to be far more complex molecules and hence, it has been challenging to arrive at their formulations with excipients that can maintain the product stability.

Thus, HER2 antibody formulations for drug delivery routes such as subcutaneous, intravenous and intramuscular delivery need further improvements like better stability for example at various temperatures and pH for solving long unaddressed need of providing a composition that can be formulated into suitable dosage form that can be stored or carried by a patient at an ordinary prevalent temperature without having need to store under controlled temperature or refrigeration.

Thus, there is an unmet need to provide compositions comprising HER2 antibody useful for treating cancer that can overcome the deficiencies associated with the compositions and formulations of known arts as mentioned above at least in respect of stability without using antioxidant, example amino acid(s) and possesses one or more advantages such as the composition or formulation that could be easily manufactured, scalable, economical, and have stabilities allowing the formulation to be stored at ordinary prevalent temperatures.

### OBJECTS OF THE INVENTION

An object of the present invention is to provide pharmaceutical composition comprising HER2 Antibody, its analogues or complexes that can satisfy the existing needs and/or can generally overcome one or more deficiencies found in the existing art.

Another object of the present invention is to provide pharmaceutical formulation comprising HER2 Antibody, its analogues or complexes that can satisfy the existing needs and/or can generally overcome one or more deficiencies found in the existing art.

One more object of the present invention is to provide process for preparation of pharmaceutical formulation comprising HER2 Antibody, its analogues or complexes that can satisfy the existing needs and/or can generally overcome one or more deficiencies found in the existing art.

Yet another object of the present invention is to provide liquid pharmaceutical formulation comprising HER2 Antibody, its analogues or complexes that can satisfy the existing needs and/or can generally overcome one or more deficiencies found in the existing art.

Still another object of the present invention is to provide process for preparation of liquid pharmaceutical formulation comprising HER2 Antibody, its analogues or complexes that can satisfy the existing needs and/or can generally overcome one or more deficiencies found in the existing art.

### SUMMARY OF THE INVENTION

This summary is provided to introduce a selection of concepts in a simplified form that are further described below in detailed description section. This summary is not intended to identify key features or essential features of the claimed subject matter, nor is it intended to be used as an aid in determining the scope of the claimed subject matter.

The present invention relates to pharmaceutical compositions comprising HER2 Antibody.

In an aspect, the present invention provides pharmaceutical compositions comprising HER2 Antibody, analogues or complexes thereof that can satisfy the existing needs and/or can generally overcome one or more deficiencies found in the existing art.

In one aspect, the present invention provides a pharmaceutical composition comprising HER2 Antibody, analogues or complexes thereof and at least one pharmaceutically acceptable excipient.

In another aspect, the present disclosure provides a pharmaceutical composition comprising:
a) a HER2 antibody;
b) a buffer;
c) a stabilizer; and
d) a surfactant.

In another aspect, the present disclosure provides a pharmaceutical composition comprising:
a) a HER2 antibody selected from but not limited to pertuzumab and trastuzumab, analogues or complexes thereof;
b) a buffer;
c) a stabilizer; and
d) a surfactant.

The buffer can be selected from the group consisting of but not limited to succinic acid, succinate, phosphate, acetate, citrate, tromethamine (Tris), sodium phosphate, citro phosphate, sodium phosphate dibasic dehydrate, tromethamine (Tris) acetate, tromethamine (Tris) phosphate, glutamic acid, glycine, histidine, histidine hydrochloride, arginine, or combinations thereof.

The stabilizer can be selected from the group consisting of but not limited to sucrose, glycerol, propylene glycol, mannitol, sorbitol, PEG 400, trehalose or any combination thereof.

The surfactant can be selected from the group consisting of but not limited to polysorbate 20, polysorbate 80, tween, polyethylene glycol, sodium dodecyl sulphate (SDS), poloxamer or any combinations thereof.

In one specific aspect, the present disclosure provides a pharmaceutical composition comprising:
a) a HER2 antibody selected from pertuzumab and trastuzumab;
b) succinic acid;
c) sucrose; and
d) Polysorbate 20.

In one aspect, the present invention provides a pharmaceutical formulation comprising the pharmaceutical composition in accordance with the present disclosure.

In one aspect, the present invention provides a liquid formulation comprising the pharmaceutical composition in accordance with the present disclosure.

In still another aspect, the present invention provides a liquid formulation comprising the pharmaceutical composition in accordance with the present disclosure, wherein the liquid composition is stable at various temperatures.

In one specific aspect, the pharmaceutical formulation is in the form of a preparation for injection.

In another aspect, the present invention provides a process for preparing the pharmaceutical formulation comprising HER2 Antibody, analogues or complexes thereof.

In another aspect, the present invention provides a process for preparing the liquid formulation comprising HER2 Antibody, analogues or complexes thereof.

In another aspect, the present invention provides to a process for preparing HER2 antibody preparation for injection, said process comprising:
a) dissolving a buffering agent, a stabilizer and a surfactant in water for injection to obtain a buffer solution;
b) dissolving a HER2 antibody into the buffer formulation solution of step (a) to obtain a bulk solution; and
c) adjusting pH of formulated bulk solution of step (b) with a pH-adjusting agent to a pH range from about 4.7 to about 6.5.

Various objects, features, aspects and advantages of the inventive subject matter will become more apparent from the following detailed description of preferred embodiments.

### BRIEF DESCRIPTION OF DRAWINGS THE INVENTION

The following drawings form part of the present specification and are included to further illustrate aspects of the present disclosure. The disclosure may be better understood by reference to the drawings in combination with the detailed description of the specific embodiments presented herein.

FIG. 1 is a graph depicting a pH profile of pertuzumab formulations F03, F04, F05, F06, F07 and F08 under stress condition (40° C.) in accordance with exemplary embodiments of the present disclosure.

FIG. 2 is a graph depicting a Protein concentration profile by OD 280 data of pertuzumab formulations F03, F04, F05, F06, F07 and F08 under stress condition (40° C.).

FIG. 3 is a graph depicting an Osmolality profile of pertuzumab formulations F03, F04, F05, F06, F07 and F08 under stress condition (40° C.).

FIG. 4 depicts a SE-HPLC % monomer profile of pertuzumab formulations F03, F04, F05, F06, F07 and F08 under stress condition (40° C.).

FIG. 5 depicts a SE-HPLC % HMW purity profile of pertuzumab formulations F03, F04, F05, F06, F07 and F08 under stress condition (40° C.).

FIG. 6 depicts a SE-HPLC % LMW purity profile of pertuzumab formulations F03, F04, F05, F06, F07 and F08 under stress condition (40° C.).

FIG. 7 depicts a CEX-HPLC % Monomer purity profile of pertuzumab formulations F03, F04, F05, F06, F07 and F08 under stress condition (40° C.).

FIG. 8 depicts a CEX-HPLC % Acidic impurity profile of pertuzumab formulations F03, F04, F05, F06, F07 and F08 under stress condition (40° C.).

FIG. 9 depicts a CEX-HPLC % Basic impurity profile of pertuzumab formulations F03, F04, F05, F06, F07 and F08 under stress condition (40° C.).

FIG. 10 depicts a RP-HPLC % monomer peak profile of pertuzumab formulations F03, F04, F05, F06, F07 and F08 under stress condition (40° C.).

FIG. 11 depicts a RP-HPLC % pre-peak profile of pertuzumab formulations F03, F04, F05, F06, F07 and F08 under stress condition (40° C.).

FIG. 12 depicts a RP-HPLC % post-peak profile of pertuzumab formulations F03, F04, F05, F06, F07 and F08 under stress condition (40° C.).

FIG. 13 depicts a pH profile of pertuzumab formulations F09, F10 and F11 under stress condition (40° C.).

FIG. 14 depicts a protein concentration profile by OD 280 data of pertuzumab formulations F09, F10 and F11 under stress condition (40° C.).

FIG. 15 depicts an osmolality profile of pertuzumab formulations F09, F10 and F11 under stress condition (40° C.).

FIG. 16 depicts a SE-HPLC % monomer profile of pertuzumab formulations F09, F10 and F11 under stress condition (40° C.).

FIG. 17 is a SE-HPLC % HMW purity profile of pertuzumab formulations F09, F10 and F11 under stress condition (40° C.).

FIG. 18 depicts a SE-HPLC % LMW purity profile of pertuzumab formulations F09, F10 and F11 under stress condition (40° C.).

FIG. 19 depicts a CEX-HPLC % monomer purity profile of pertuzumab formulations F09, F10 and F11 under stress condition (40° C.).

FIG. 20 depicts a CEX-HPLC % acidic impurity profile of pertuzumab formulations F09, F10 and F11 under stress condition (40° C.).

FIG. 21 depicts a CEX-HPLC % Basic impurity profile of pertuzumab formulations F09, F10 and F11 under stress condition (40° C.).

FIG. 22 depicts a CEX-HPLC graph of rate of degradation for % acidic impurity profile of pertuzumab formulations F09 and F10 under stress condition (40° C.).

FIG. 23 depicts a CEX-HPLC graph of rate of degradation for % main peak of pertuzumab formulations F09 and F10 under stress condition (40° C.).

### DETAILED DESCRIPTION

The following is a detailed description of embodiments of the disclosure. The embodiments are in such detail as to clearly communicate the disclosure. However, the amount of detail offered is not intended to limit the anticipated variations of embodiments: on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, the numbers expressing quantities of ingredients, properties such as concentration, reaction conditions, and so forth, used to describe and claim certain embodiments of the invention are to be understood as being modified in some instances by the term “about”. The term “about” in some embodiments is ±5%. Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a.” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

Various terms as used herein are shown below. To the extent a term used in a claim is not defined below, it should be given the broadest definition persons in the pertinent art have given that term as reflected in printed publications and issued patents at the time of filing.

### Definitions

As referred to herein the term “antibody” includes whole antibodies and any antigen-binding fragment (i.e., “antigen-binding portion”) or single chains thereof. An “antibody” refers to a glycoprotein comprising at least two heavy (H) chains and two light (L) chains interconnected by disulphide bonds, or an antigen-binding portion thereof.

Pertuzumab is an antibody and pertuzumab as referred herein includes amino acid sequences of full-length heavy and full-length light chains or variable regions of heavy and light chains of pertuzumab or antigen binding portion of pertuzumab.

As used herein the term “Pharmaceutical composition” refers to the combination of one or more drug substances and one or more excipients”.

As used herein the term “Drug product,” “pharmaceutical dosage form,” “dosage form.” “final dosage form” and the like, refer to a pharmaceutical formulation that is administered to a subject in need of treatment and generally may be in the form of tablets, capsules, sachets containing powder or granules, pellets, liquid solutions or suspensions, patches, or the like.

As used herein the term ‘pharmaceutically acceptable excipient’ means, but not limited to, any inactive ingredient which is required for the formulation of pertuzumab in a suitable dosage form. Particularly the excipient includes, but not limited to, diluents, carriers, fillers, bulking agents, binders, disintegrants, polymer, lubricant, glidant, surface active agents, stabilizers, absorption accelerators, flavoring agents, preservatives, antioxidants, buffering agents, and any other excipient commonly used in the pharmaceutical industry.

As used herein the term ‘buffer’ means, but is not limited to an aqueous solution consisting of a mixture of a weak acid and its conjugate base, or vice versa. Its pH changes very little when a small amount of strong acid or base is added to it. Particularly the buffer includes, but is not limited to, histidine, histidine hydrochloride, phosphate, succinate, citrate, tromethamine (Tris), arginine, sodium phosphate and glycine, sodium phosphate dibasic dehydrate, tromethamine (Tris) acetate, tromethamine (Tris) phosphate, glutamic acid, citro phosphate, acetate and combination thereof.

As used herein the term ‘stabilizer’ means but is not limited to an agent which provides stability to the formulation. Particularly the stabilizer includes, but is not limited to, glycerol, sucrose, propylene glycol, mannitol, sorbitol, PEG 400 and trehalose or combinations thereof.

As used herein the term ‘surfactant’ means but is not limited to a substance or a chemical that is added to lower the surface tension (or interfacial tension) between two liquids Particularly the surfactant includes, but is not limited to, polysorbate 20, polysorbate 80, tween, polyethylene glycol, sodium dodecyl sulphate (SDS), poloxamer or any combination thereof.

As used herein the term “Subject” includes humans, non-human mammals (e.g., dogs, cats, rabbits, cattle, horses, sheep and the like) or non-mammals (e.g., birds and the like).

The present invention relates to pharmaceutical compositions comprising HER2 Antibody.

In an embodiment, the present invention provides pharmaceutical compositions comprising HER2 Antibody, analogues or complexes thereof.

In one embodiment, the present invention provides a pharmaceutical formulation comprising HER2 Antibody, analogues or complexes thereof.

In one embodiment, the present invention provides a pharmaceutical composition comprising HER2 Antibody, analogues or complexes thereof and at least one pharmaceutically acceptable excipient.

In another embodiment, the present invention provides a pharmaceutical composition comprising:
a) a HER2 antibody;
b) a buffer;
c) a stabilizer; and
d) a surfactant.

In another embodiment, the present invention provides a pharmaceutical composition comprising:
a) a HER2 antibody is selected from but not limited to pertuzumab and trastuzumab, analogues or complexes thereof;
b) a buffer;
c) a stabilizer; and
d) a surfactant.

The pharmaceutical composition comprising a HER2 antibody selected from pertuzumab and trastuzumab, wherein the pH of the composition ranges from about 4.7 to about 6.5, preferably from about 4.9 to about 6.3.

The buffer can be selected from the group consisting of but not limited to succinic acid, succinate, phosphate, acetate, citrate, tromethamine (Tris), sodium phosphate, citro phosphate, sodium phosphate dibasic dehydrate, tromethamine (Tris) acetate, tromethamine (Tris) phosphate, glutamic acid, glycine, histidine, histidine hydrochloride, arginine, or combinations thereof.

The stabilizer can be selected from the group consisting of but not limited to sucrose, glycerol, propylene glycol, mannitol, sorbitol, PEG 400, trehalose or any combination thereof.

The surfactant can be selected from the group consisting of but not limited to polysorbate 20, polysorbate 80, tween, polyethylene glycol, sodium dodecyl sulphate (SDS), poloxamer or any combinations thereof.

In one embodiment, the HER2 antibody selected from pertuzumab and trastuzumab, analogues or complexes thereof is in an amount ranging from about 10 mg/ml to 100 mg/ml.

In one embodiment, the buffer is in an amount ranging from about 3 mM to about 100 mM. More preferably, the amount of buffer is selected from about 3 mM to about 60 mM.

In one embodiment, the buffer is present in a concentration ranging from about 0.01% to about 10%, preferably, from about 0.02% to about 8.0% w/w of the composition.

In one embodiment, the stabilizer is present in the concentration ranging from about 0.01% to about 10% w of the composition. Preferably, the concentration of stabilizer is from about 0.02% to about 8% w of the composition.

In one specific embodiment, the stabilizer is present in an amount ranging from about 10 mg/ml to about 110 mg/ml of the composition. More preferably, the stabilizer is present in the concentration ranging from about 20 mg/ml to about 100 mg/ml of the composition.

In one embodiment, the surfactant is present in a concentration ranging from about 0.01% w/v to about 10% w/v of the composition. More preferably, the concentration of surfactant is from about 0.01% w/v to about 5% w/v of the composition.

In one specific embodiment, the surfactant is present in an amount ranging from about 0.1 mg/ml to about 0.5 mg/ml w/v of the composition.

In one embodiment, the present invention provides a pharmaceutical composition comprising:
a) a HER2 antibody selected from pertuzumab or trastuzumab, analogues or complexes thereof in an amount from about 10 mg/ml to about 100 mg/ml;
b) a buffer in an amount from about 5 mM to about 30 mM;
c) a stabilizer in an amount from 20 mg/ml to about 100 mg/ml; and
d) a surfactant in an amount from 0.1 mg/ml to about 0.5 mg/ml.

In one specific embodiment, the present disclosure provides a pharmaceutical composition comprising:
a) a HER2 antibody selected from pertuzumab and trastuzumab;
b) a buffer, succinic acid;
c) a stabilizer, sucrose; and
d) a surfactant, Polysorbate 20.

The pharmaceutical compositions comprising HER2 Antibody according to the present invention are particularly useful for the treatment of disease(s) or disorder(s) which are particularly acute in nature and which require a short term but mild to moderate treatment, or even some chronic conditions which favorably respond to or are alleviated by the pharmaceutical compositions comprising HER2 Antibody. The pharmaceutical compositions comprising HER2 Antibody are useful prophylactically or therapeutically depending upon the pathological condition intended to be prevented or treated respectively.

In one embodiment pharmaceutical compositions comprising HER2 Antibody of the present invention are useful in the prophylaxis, amelioration and/or treatment of breast cancer in a subject in need thereof.

In another embodiment pharmaceutical compositions comprising HER2 antibody of the present invention are useful in the prophylaxis, amelioration and/or treatment of Breast Cancer, which comprises administrating to a subject in need thereof a pharmaceutically effective amount of HER2 antibody composition.

An embodiment of the present invention relates to methods of using pharmaceutical compositions comprising HER2 Antibody of the present invention for the prophylaxis, amelioration and/or treatment of Breast Cancer, which comprises administrating to a subject in need thereof a pharmaceutically effective amount of composition comprising HER2 Antibody.

In yet another embodiment, the pharmaceutical compositions comprising HER2 Antibody of the present invention are useful in the treatment of the aforementioned diseases, disorders and conditions in combination with another disease-modifying drug. The pharmaceutical compositions comprising HER2 Antibody may be used in combination with one or more other drugs in the treatment, prevention, suppression or amelioration of diseases or conditions for which pharmaceutical compositions comprising HER2 Antibody may have utility, where the combination with the other drugs may be safer or more effective than the antibody and the drug alone.

A further embodiment of the present invention is the use of a pharmaceutical compositions comprising HER2 Antibody for the manufacture of a medicament for the prophylaxis, amelioration and/or treatment of breast cancer, involving HER2 Antibody in a subject in need thereof.

In still another embodiment of the present invention is the pharmaceutical compositions comprising HER2 Antibody for use as a medicament for the prophylaxis, amelioration and/or treatment of breast cancer, involving HER2 Antibody, in a subject in need thereof preferably a mammal including a human.

Pharmaceutical compositions containing a HER2 Antibody according to the present invention may be administered parenterally to patients in need of such a treatment. Parenteral administration may be performed by subcutaneous, intramuscular or intravenous injection by means of a syringe, optionally a pen-like syringe. Alternatively, parenteral administration can be performed by means of an infusion pump. A further option is a composition which may be a solution or suspension for the administration of the HER2 Antibody in the form of a nasal or pulmonal spray. As a still further option, the pharmaceutical compositions containing the HER2 Antibody of the invention can also be adapted for transdermal administration, for example in the form of a patch, optionally a iontophoretic patch, or transmucosal for example for buccal administration.

The pharmaceutical compositions comprising HER2 Antibody may be administered in the form of any pharmaceutical formulation.

In one embodiment, the present invention provides a pharmaceutical formulation comprising the pharmaceutical composition in accordance with the present disclosure and a process for preparing the same.

In one embodiment, the present invention provides a pharmaceutical formulation comprising:
a) a HER2 antibody selected from pertuzumab or trastuzumab, analogues or complexes thereof in an amount from about 10 mg/ml to about 100 mg/ml;
b) a buffer in an amount from about 5 mM to about 30 mM;
c) a stabilizer in an amount from 20 mg/ml to about 100 mg/ml; and
d) a surfactant in an amount from 0.1 mg/ml to about 0.5 mg/ml.

In one embodiment, the present invention provides a pharmaceutical formulation comprising:
a) a HER2 antibody selected from pertuzumab or trastuzumab, analogues or complexes thereof;
b) a buffer, succinic acid;
c) a stabilizer, sucrose; and
d) a surfactant, Polysorbate 20.

The pharmaceutical formulation of the present invention can be manufactured by the processes well known in the art, for example, by means of conventional mixing, dissolving, dry granulation, wet granulation, dragee-making, levitating, emulsifying, encapsulating, entrapping, lyophilizing processes or spray drying.

In one embodiment, the present invention provides a liquid formulation comprising HER2 Antibody, analogues or complexes thereof.

In another embodiment, the present invention provides a process for preparing the liquid formulation comprising HER2 Antibody, analogues or complexes thereof.

In one specific embodiment, the liquid formulation is in the form of a preparation for injection.

In another embodiment, the present invention provides to a process for preparing HER2 antibody preparation for injection, said process comprising:
a) dissolving a buffering agent, a stabilizer and a surfactant in water for injection to obtain a buffer solution;
b) dissolving a HER2 antibody into the buffer formulation solution of step (a) to obtain a bulk solution; and
c) adjusting pH of formulated bulk solution of step (b) with a pH-adjusting agent to a pH range from about 4.7 to about 6.5.

In one embodiment, the pH is maintained at about 4.7 to 6.5, more preferably at 4.9 to 6.3.

The buffering agent comprises succinic acid.

The stabilizer comprises sucrose.

The surfactant comprises Polysorbate 20.

In an embodiment, the pH-adjusting agent is selected from hydrochloric acid, sodium hydroxide using concentration ranging from about 0.1N to about 5N.

In still another embodiment, the present invention provides a liquid formulation comprising HER2 Antibody, analogues or complexes thereof, wherein the liquid formulation is stable at various temperatures.

In general, the pharmaceutical formulation is found to be physically unstable when it exhibits turbidity. Physical stability of the formulations is evaluated by means of visual inspection and turbidity after storage of the formulation at different temperatures in top filled glass cartridges for various time periods. Visual inspection of the formulations is performed in a sharp focused light with a dark background. A formulation is classified physical unstable with respect to protein aggregation, when it shows visual turbidity in daylight.

In one embodiment, the pharmaceutical formulation according to the present invention is physically stable for a period of about 10-25 days at about 40° C.

In one embodiment, the pharmaceutical formulation according to the present invention is physically stable for a period up to about 6 months at about 2° C.-about 8° C.

In one embodiment, the pharmaceutical formulation according to the present invention is physically stable for a period of up to about 6 months at about 25° C.

In one embodiment, the pharmaceutical formulation according to the present invention is found to be stable with no significant change in pH for a period of up to about 6 months at about 25° C.

In one embodiment, the pharmaceutical formulation according to the present invention is found to be stable with no significant change in purity of the active drug substance that is the HER2 Antibody for a period of up to about 6 months at about 25° C.

In one embodiment, the pharmaceutical formulation according to the present invention is found to be stable with no significant change in the content of the active drug substance that is the HER2 Antibody for a period of up to about 6 months at about 25° C.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

The present invention is further explained in the form of following examples. However, it is to be understood that the following examples are merely illustrative and are not to be taken as limitations upon the scope of the invention.

### Abbreviations

The abbreviations used in present application are defined below:
SE HPLC: Size Exclusion High performance liquid chromatography
CEX HPLC: Cation Exchange High performance liquid chromatography
RP HPLC: Reverse phase High performance liquid chromatography
HMW: High molecular weight
LMW: Low molecular weight
%: percentage
Tm: melting temperature
mg/mL: milligram/milliliter:
degree Celsius
mM: milli molar

### Example 1

Pertuzumab Formulations with Different Buffers

TABLE 1
Compositions of different Formulations

| Formulation | Buffer | Stabilizer | Surfactant |
|---|---|---|---|
| F03&F09 | Histidine acetate | Sucrose | Polysorbate 20 |
| | (20 mM) | (41.1 mg/mL) | (0.2 mg/mL) |
| F04&F10 | Succinate (10 mM) | Sucrose | |
| | | (41.1 mg/mL) | |
| F05 | Phosphate (10 mM) | Sucrose | |
| | | (41.1 mg/mL) | |
| F06 | Histidine hydrochloride | Sucrose | |
| | (10 mM) | (41.1 mg/mL) | |
| F07&F11 | Acetate (10 mM) | Sucrose | |
| | | (100 mg/mL) | |
| F08 | Acetate (10 mM) | Sucrose | |
| | | (41.1 mg/mL) | |

### I. Formulations Set 1

Specific formulations denoted as Formulations Set 1, with compositions as mentioned in Table 2 were formulated comprising different buffers by diluting drug substance of 80 mg/Ml in different buffer matrix to achieve target protein concentration. Thermal profiles of these formulation were studied using differential scanning calorimetry (DSC).

TABLE 2
Formulations set 1

| Buffers | Concentration (mM) | pH |
|---|---|---|
| Histidine acetate | 20 | 6.0 |
| Histidine hydrochloride | 10 | 6.0 |
| Succinate | 10 | 6.0 |
| Phosphate | 10 | 6.0 |
| Citrate | 10 | 6.0 |
| Citro-phosphate | 10 | 6.0 |
| Acetate | 10 | 5.2 |

TABLE 3
Thermal profile observed with different buffers at different time
interval at stress conditions

| Buffers | Tm1 (° C.) | Tm2 (° C.) | Tm3 (° C.) |
|---|---|---|---|
| Histidine acetate | 70.49 | 84.47 | — |
| Histidine hydrochloride | 70.54 | 84.18 | — |
| Histidine acetate | 70.49 | 84.47 | — |
| Succinate | 71.24 | 81.12 | — |
| Phosphate | 72.43 | 82.04 | — |
| Citrate | 71.04 | 79.76 | 85.35 |
| Citro-phosphate | 70.71 | 80.32 | — |
| Acetate | 70.85 | 85.30 | — |

### Observation:

Tm profile for all the buffer components were studied. Citrate and citro phosphate buffers due to their thermal profiles showed lower Tm and hence were not selected for further experiments. Tm1 observed for succinate buffer was higher than histidine acetate and histidine HCl buffer, hence succinate and acetate buffers were selected for further studies.

### II. Formulations Set 2

Specific formulations denoted as Formulations Set 2, with compositions as mentioned in Table 4 were formulated comprising different concentration of stabilizer. Buffer exchange of drug substance was done in the respective buffers and formulations F03, F04, F05 and F06 were prepared of 30 mg/mL. For F07 and F08 formulations, dilutions using respective formulation buffer were done in DS to make 30 mg/mL formulation. Prepared formulations were filled in USP type-1 glass vials and stoppered with bromobutyl rubber stopper. Filled vials (30 mg/mL drug product) were charged for stress stability at 40° C.±2° C./65±5% RH for 28 days. Concentration of stabilizer i.e., sucrose in formulation was varied from 4.1% to 10%. The concentration of the stabilizer and osmolality associated with it is mentioned in Table 5. Stability study of each formulation by pH, osmolality, protein content, SE HPLC, CEX HPLC, RP HPLCat different time interval under stress condition of temperature at 40° C. was performed.

TABLE 4
Formulations set 2

| Formulation | Buffer | Stabilizer | Surfactant |
|---|---|---|---|
| F03 | Histidine acetate | Sucrose | Polysorbate 20 |
| | (20 mM) | (41.1 mg/mL) | (0.2 mg/mL) |
| F04 | Succinate (10 mM) | Sucrose | |
| | | (41.1 mg/mL) | |
| F05 | Phosphate (10 mM) | Sucrose | |
| | | (41.1 mg/mL) | |
| F06 | Histidine hydrochloride | Sucrose | |
| | (10 mM) | (41.1 mg/mL) | |
| F07 | Acetate (10 mM) | Sucrose | |
| | | (100 mg/mL) | |
| F08 | Acetate (10 mM) | Sucrose | |
| | | (41.1 mg/mL) | |

TABLE 5
Stabilizer concentration and osmolality

| Stabilizer | Concentration (mg/ml) | Osmolality (mOsm/kg) |
|---|---|---|
| Sucrose | 41.1 | 126 |
| Sucrose | 100 | 325 |

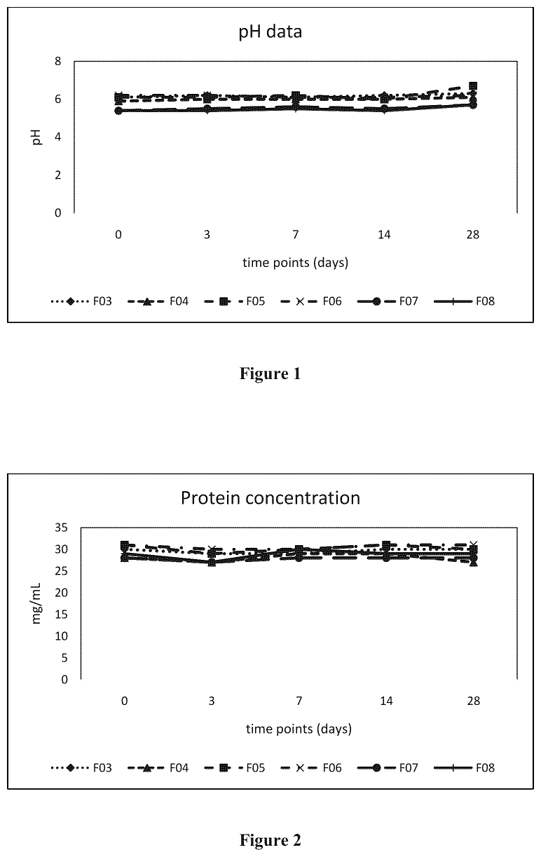

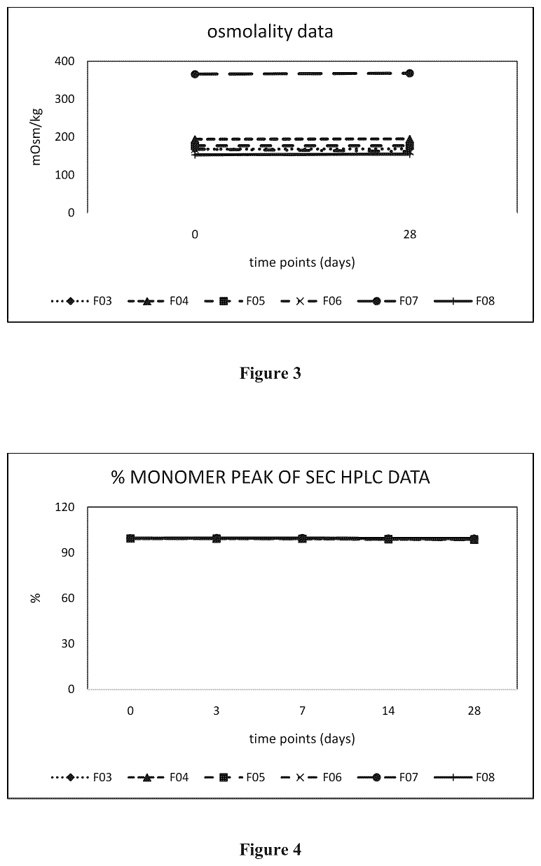

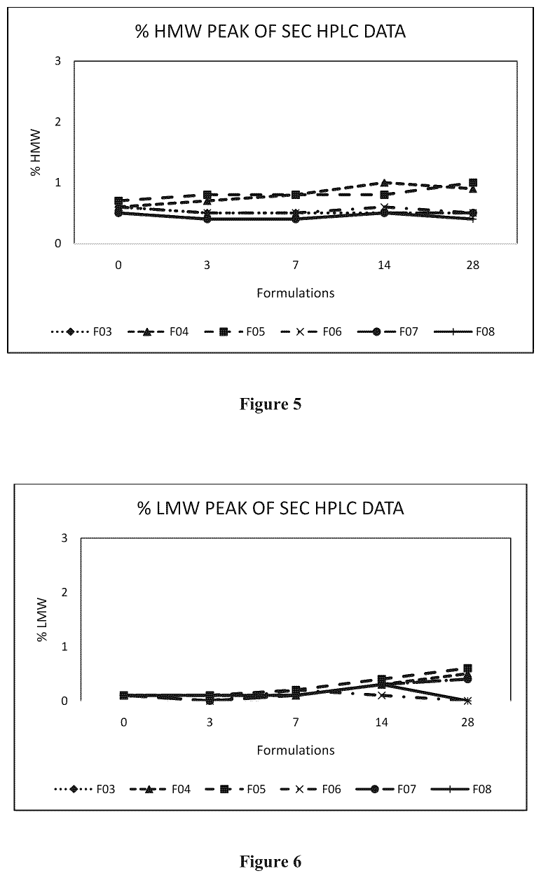

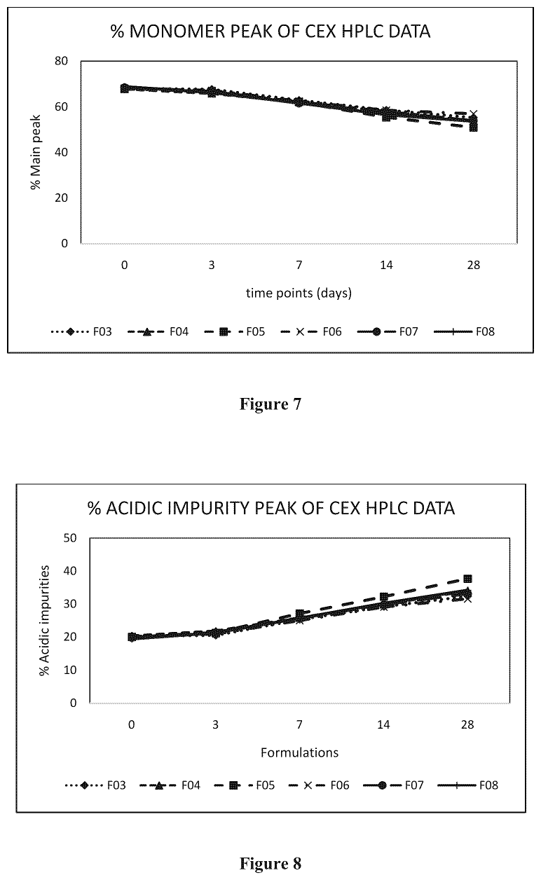

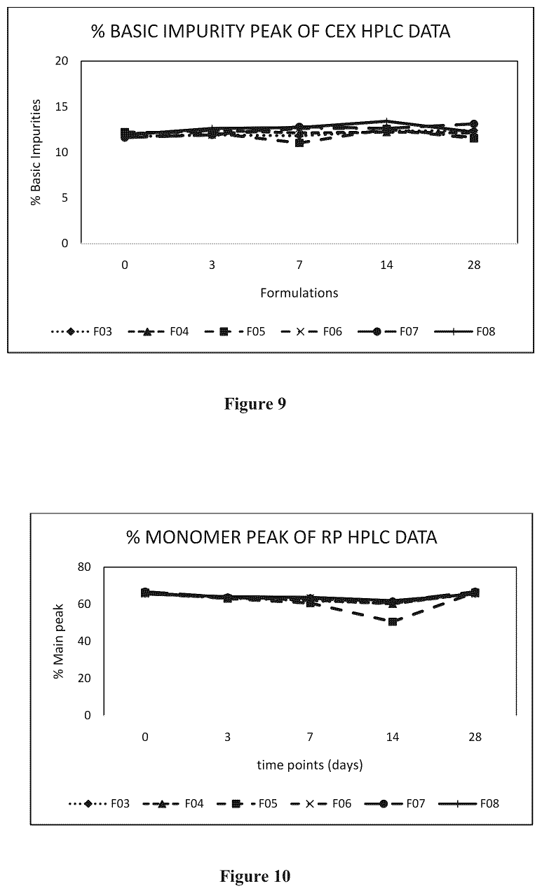

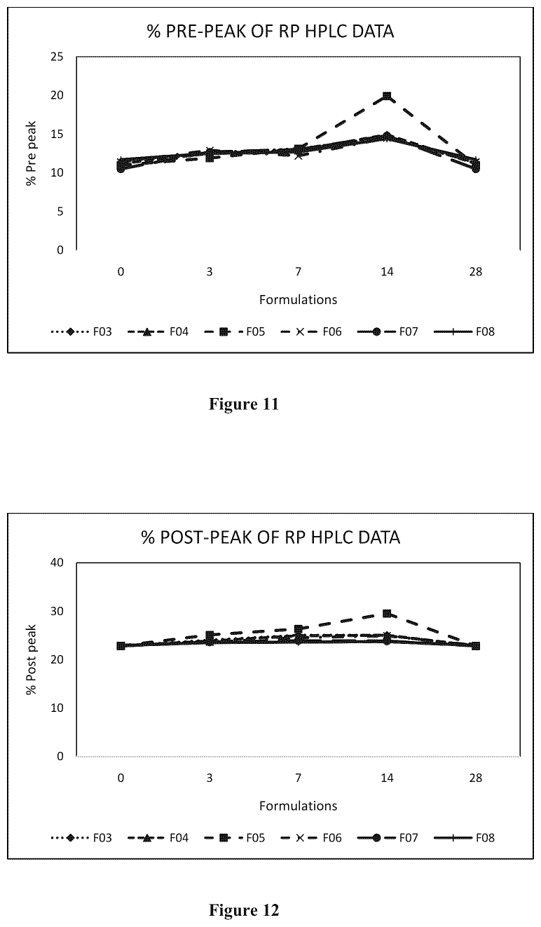

Results of pH, osmolality, protein content, size-exclusion high-performance liquid chromatography (SE HPLC), cation-exchange exclusion high-performance liquid chromatography (CEX HPLC), reverse phase high-performance liquid chromatography (RP HPLC) analysis of different formulations in formulation set 2 at different time intervals under stress condition at 40° C. have been described below in Table 6 to Table 17 and illustrated by FIG. 1 to FIG. 12.

### A. pH

TABLE 6
pH profile of pertuzumab formulations F03, F04, F05,
F06, F07 and F08 under stress condition (40° C.)

| Time points | Formulations | | | | | |
|---|---|---|---|---|---|---|
| (Days) | F03 | F04 | F05 | F06 | F07 | F08 |
| 0 | 6.1 | 5.9 | 6.1 | 6.2 | 5.4 | 5.4 |
| 3 | 6.2 | 6.0 | 6.1 | 6.2 | 5.5 | 5.4 |
| 7 | 6.1 | 6.0 | 6.2 | 6.1 | 5.6 | 5.5 |
| 14 | 6.2 | 6.0 | 6.0 | 6.1 | 5.5 | 5.4 |
| 28 | 6.3 | 6.1 | 6.7 | 6.2 | 5.7 | 5.7 |

### Observation:

No change in pH was observed for all formulations till day 14. At 28 days, formulation F05, F07 and F08 showed increase in pH from initial.

### B. Protein Concentration:

TABLE 7
Protein concentration profile by OD 280 data
of pertuzumab formulations F03, F04, F05, F06,
F07 and F08 under stress condition (40° C.)

| Time points | Formulations | | | | | |
|---|---|---|---|---|---|---|
| (Days) | F03 | F04 | F05 | F06 | F07 | F08 |
| 0 | 30 | 28 | 31 | 31 | 28 | 29 |
| 3 | 29 | 27 | 29 | 30 | 27 | 27 |
| 7 | 29 | 29 | 30 | 30 | 28 | 30 |
| 14 | 30 | 29 | 31 | 31 | 28 | 29 |
| 28 | 30 | 27 | 30 | 31 | 28 | 29 |

### Observation:

No change in protein concentration was observed for all formulations till day 28. Concentration was between 27 to 31 mg/mL throughout the stability study.

### C. Osmolality:

TABLE 8
Osmolality purity profile of pertuzumab formulations F03,
F04, F05, F06, F07 and F08 under stress condition (40° C.)

| Time points | Formulations | | | | | |
|---|---|---|---|---|---|---|
| (Days) | F03 | F04 | F05 | F06 | F07 | F08 |
| 0 | 168 | 194 | 177 | 168 | 366 | 153 |
| 28 | 169 | 195 | 177 | 162 | 368 | 155 |

### Observation:

No change in osmolality was observed for all formulations till day 28, except formulation F06 containing histidine HCl buffer, where decrease in osmolality was observed.

### D. SE HPLC

TABLE 9
SE- HPLC % monomer profile of pertuzumab formulations F03,
F04, F05, F06, F07 and F08 under stress condition (40° C.)

Formulations

| Days | F03 | F04 | F05 | F06 | F07 | F08 |
|---|---|---|---|---|---|---|
| 0 | 99.3 | 99.3 | 99.2 | 99.3 | 99.4 | 99.4 |
| 3 | 99.5 | 99.2 | 99.1 | 99.5 | 99.5 | 99.5 |
| 7 | 99.4 | 99.1 | 99.0 | 99.3 | 99.5 | 99.5 |
| 14 | 99.2 | 98.7 | 98.8 | 99.1 | 99.3 | 99.3 |
| 28 | 99.1 | 98.7 | 98.4 | 99.0 | 99.1 | 99.2 |

TABLE 10
SE-HPLC % HMW purity profile of pertuzumab formulations F03,
F04, F05, F06, F07 and F08 under stress condition (40° C.)

Formulations

| Days | F03 | F04 | F05 | F06 | F07 | F08 |
|---|---|---|---|---|---|---|
| 0 | 0.6 | 0.6 | 0.7 | 0.6 | 0.5 | 0.5 |
| 3 | 0.5 | 0.7 | 0.8 | 0.5 | 0.4 | 0.4 |
| 7 | 0.5 | 0.8 | 0.8 | 0.5 | 0.4 | 0.4 |
| 14 | 0.5 | 1.0 | 0.8 | 0.6 | 0.5 | 0.5 |
| 28 | 0.5 | 0.9 | 1.0 | 0.5 | 0.5 | 0.4 |

TABLE 11
SE-HPLC % LMW purity profile of pertuzumab formulations F03,
F04, F05, F06, F07 and F08 under stress condition (40° C.)

Formulations

| Days | F03 | F04 | F05 | F06 | F07 | F08 |
|---|---|---|---|---|---|---|
| 0 | 0.1 | 0.1 | 0.1 | 0.1 | 0.1 | 0.1 |
| 3 | 0.1 | 0.1 | 0.1 | 0.0 | 0.0 | 0.1 |
| 7 | 0.1 | 0.1 | 0.2 | 0.2 | 0.1 | 0.1 |
| 14 | 0.3 | 0.3 | 0.4 | 0.1 | 0.3 | 0.3 |
| 28 | 0.4 | 0.5 | 0.6 | 0.0 | 0.4 | 0.4 |

### Observation:

No change in purity was observed for all formulations till day 28, except in F04 and F05 there was slight increase in % HMW and % LMW was observed, however the same is within the permissible limits.

### E. CEX HPLC

TABLE 12
CEX- HPLC % Monomer purity profile of pertuzumab formulations
F03, F04, F05, F06, F07 and F08 under stress condition (40° C.)

Formulations

| Days | F03 | F04 | F05 | F06 | F07 | F08 |
|---|---|---|---|---|---|---|
| 0 | 68.0 | 67.8 | 67.8 | 68.1 | 68.5 | 68.8 |
| 3 | 67.6 | 65.8 | 66.6 | 67.2 | 66.7 | 66.2 |
| 7 | 62.9 | 62.1 | 62.0 | 62.4 | 61.5 | 61.6 |
| 14 | 58.5 | 57.9 | 55.3 | 58.4 | 57.6 | 56.5 |
| 28 | 55.2 | 53.8 | 50.9 | 56.9 | 53.8 | 53.6 |

TABLE 131
CEX- HPLC % Acidic impurity profile of pertuzumab formulations
F03, F04, F05, F06, F07 and F08 under stress condition (40° C.)

Formulations

| Days | F03 | F04 | F05 | F06 | F07 | F08 |
|---|---|---|---|---|---|---|
| 0 | 20.3 | 20.3 | 20.0 | 20.1 | 19.8 | 19.4 |
| 3 | 20.5 | 21.8 | 21.3 | 20.9 | 21.4 | 21.3 |
| 7 | 25.3 | 25.8 | 27.1 | 25.0 | 25.7 | 25.7 |
| 14 | 29.2 | 29.9 | 32.2 | 29.1 | 29.8 | 30.2 |
| 28 | 32.4 | 34.0 | 37.7 | 31.6 | 33.1 | 34.2 |

TABLE 14
CEX- HPLC % Basic impurity profile of pertuzumab formulations
F03, F04, F05, F06, F07 and F08 under stress condition (40° C.)

Formulations

| Days | F03 | F04 | F05 | F06 | F07 | F08 |
|---|---|---|---|---|---|---|
| 0 | 11.7 | 12.0 | 12.2 | 11.8 | 11.6 | 11.9 |
| 3 | 11.9 | 12.4 | 12.1 | 11.9 | 11.9 | 12.6 |
| 7 | 11.8 | 12.1 | 11.0 | 12.6 | 12.8 | 12.7 |
| 14 | 12.3 | 12.2 | 12.5 | 12.6 | 12.6 | 13.4 |
| 28 | 12.4 | 12.1 | 11.5 | 11.6 | 13.1 | 12.2 |

No change in purity was observed for all formulations till day 28, except in F05 there was increase in % acidic impurities and decrease in % main peak, however, the same was within the permissible limits.

### F. RP HPLC:

TABLE 25
RP- HPLC % monomer peak profile of pertuzumab formulations F03,
F04, F05, F06, F07 and F08under stress condition (40° C.)

Formulation system

| Days | F03 | F04 | F05 | F06 | F07 | F08 |
|---|---|---|---|---|---|---|
| 0 | 65.8 | 66.0 | 66.2 | 65.7 | 66.7 | 65.5 |
| 3 | 63.4 | 63.6 | 63.0 | 63.2 | 63.7 | 64.1 |
| 7 | 61.9 | 62.3 | 60.6 | 63.3 | 63.1 | 63.7 |
| 14 | 60.4 | 60.2 | 50.6 | 60.6 | 61.4 | 61.9 |
| 28 | 65.8 | 66.0 | 66.2 | 65.7 | 66.7 | 65.5 |

TABLE 36
RP- HPLC % pre-peak profile of Pertuzumab formulations F03,
F04, F05, F06, F07 and F08 under stress condition (40° C.)

Formulation system

| Days | F03 | F04 | F05 | F06 | F07 | F08 |
|---|---|---|---|---|---|---|
| 0 | 11.3 | 11.2 | 11.0 | 11.4 | 10.5 | 11.7 |
| 3 | 12.6 | 12.5 | 11.9 | 12.9 | 12.7 | 12.5 |
| 7 | 13.1 | 12.9 | 13.1 | 12.2 | 13.0 | 12.7 |
| 14 | 14.6 | 14.9 | 19.9 | 14.5 | 14.8 | 14.4 |
| 28 | 11.3 | 11.2 | 11.0 | 11.4 | 10.5 | 11.7 |

TABLE 47
RP- HPLC % post-peak profile of pertuzumab formulations F03,
F04, F05, F06, F07 and F08 under stress condition (40° C.)

Formulation system

| Days | F03 | F04 | F05 | F06 | F07 | F08 |
|---|---|---|---|---|---|---|
| 0 | 22.8 | 22.8 | 22.8 | 22.9 | 22.8 | 22.9 |
| 3 | 24.0 | 23.8 | 25.1 | 23.6 | 23.6 | 23.5 |
| 7 | 25.0 | 24.9 | 26.3 | 24.5 | 23.9 | 23.6 |
| 14 | 25.0 | 25.0 | 29.5 | 24.9 | 23.8 | 23.7 |
| 28 | 22.8 | 22.8 | 22.8 | 22.9 | 22.8 | 22.9 |

### Observation:

No change in purity was observed for all formulations till day 28 and it is consistent compared with day 0.

### III. Formulation Set 3

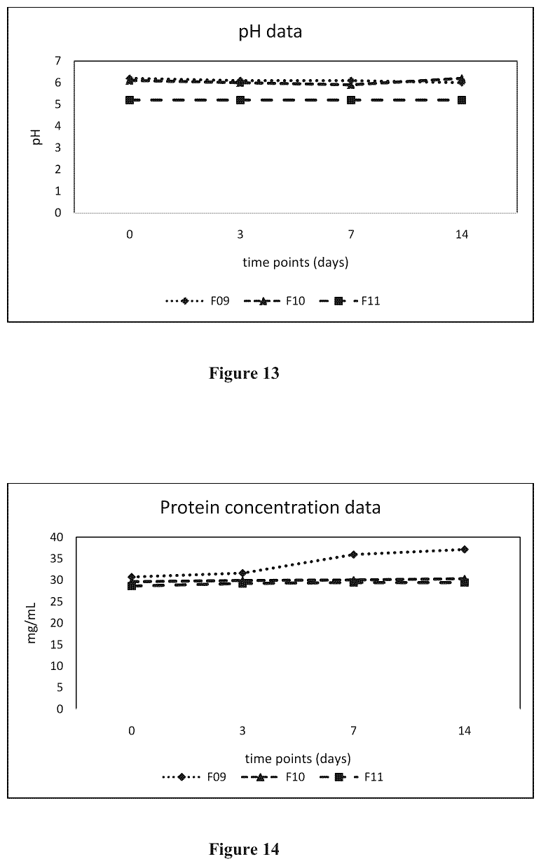

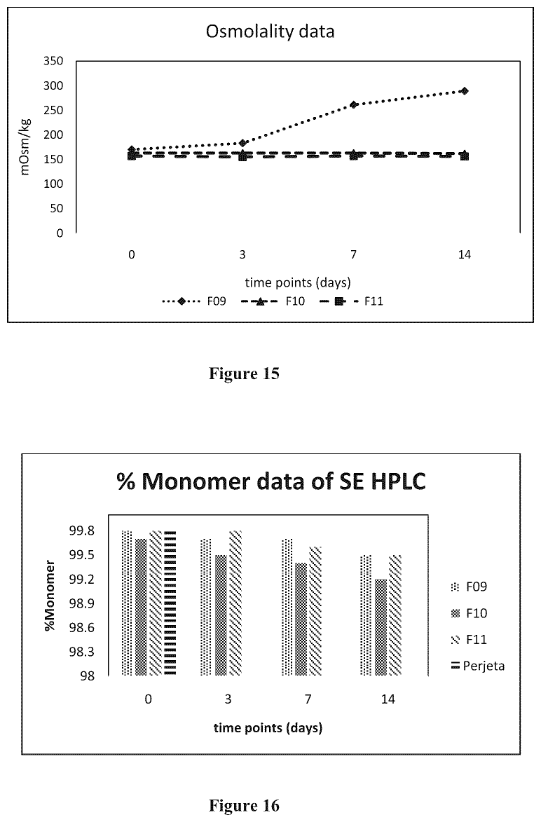

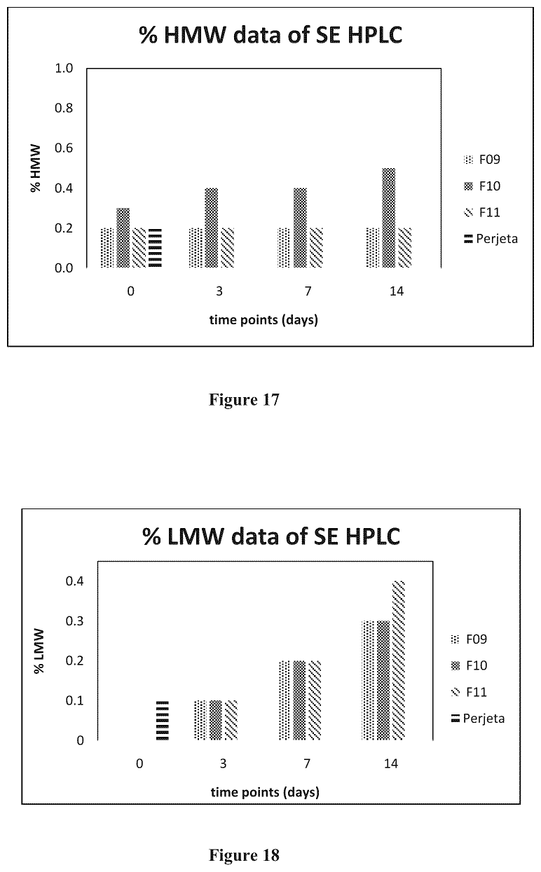

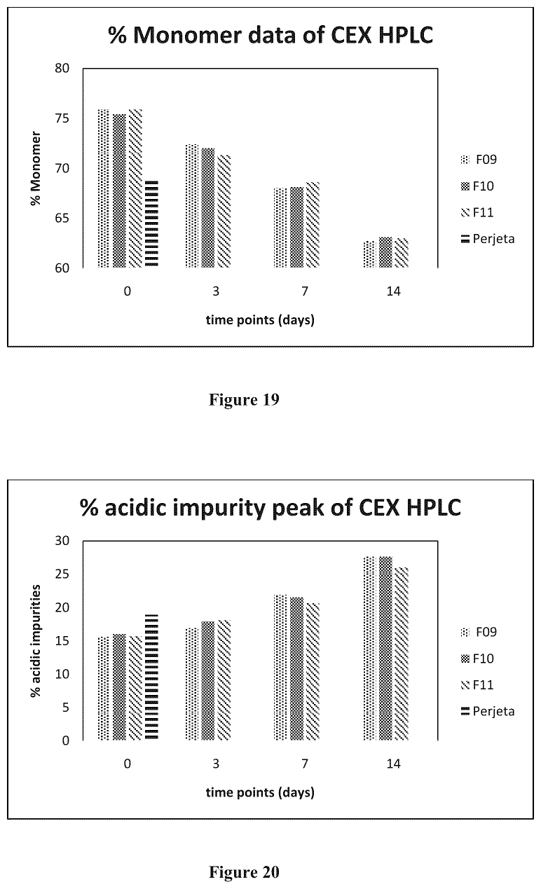

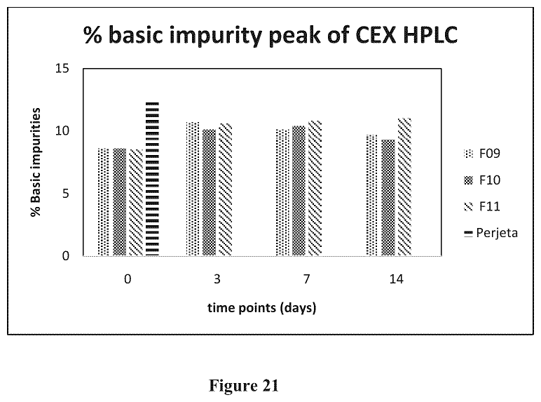

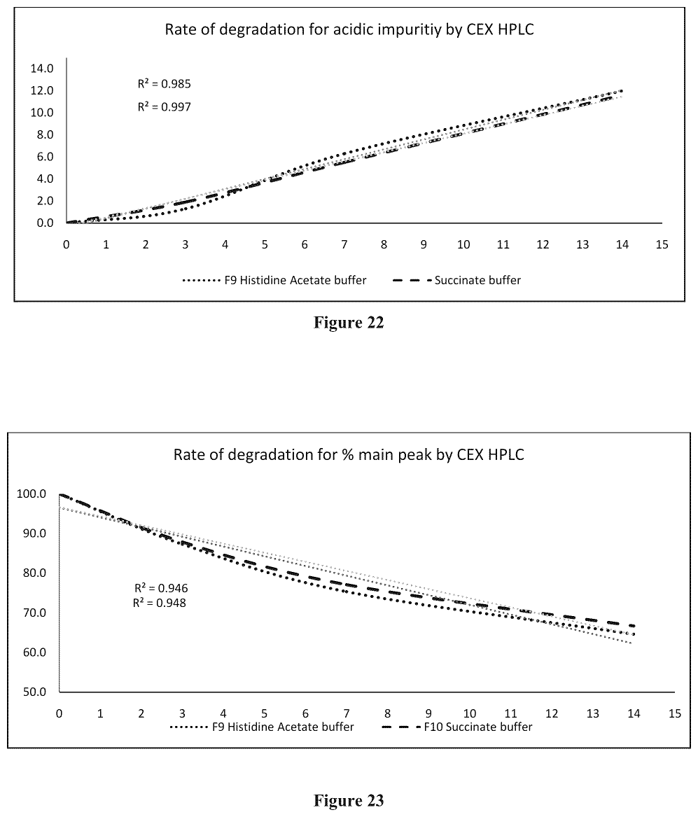

Specific formulations denoted as Formulations Set 3, with compositions as mentioned in Table 18 were formulated comprising different concentration of buffer and stabilizer. The two lead formulations selected were F04 and F07. DS of concentration approximately 85 mg/mL in F07 buffer was diluted to make 30 mg/mL formulated bulk solution and F11 formulation was prepared. F09 and F10 formulations were prepared by performing buffer exchange of DS in respective buffers. Prepared formulations were filled as 420 mg in USP type-1 glass vials and stoppered with bromobutyl rubber stopper. Concentration of stabilizer i.e. sucrose in formulation was varied from 4.1% to 10%. The Thermal profiles of these formulation were associated with it is mentioned in Table 19. Stability study of each formulation by pH, osmolality, protein content/% monomer, acidic impurity profile, basic impurity profile, rate of degradation of acidic impurity and rate of degradation of main peak/protein by SE HPLC, CEX HPLC, RP HPLCas applicable at different time interval under stress condition of temperature at 40° C. was performed. All the results are reported under Table 20 to Table 25 and FIG. 13 to FIG. 23.

TABLE 18
Buffer combination with stabilizer for stress stability 2

| Formulation | Buffer | Stabilizer | Surfactant |
|---|---|---|---|
| F09 | Histidine acetate | Sucrose | Polysorbate 20 |
| | (20 mM) | (41.1 mg/mL) | (0.2 mg/mL) |
| F10 | Succinate (10 mM) | Sucrose | |
| | | (41.1 mg/mL) | |
| F11 | Acetate (10 mM) | Sucrose | |
| | | (41.1 mg/mL) | |

TABLE 59
Tm data of stage 2 buffers

| Formulations | Tm1 (° C.) | Tm2 (° C.) |
|---|---|---|
| Histidine acetate (F09) | 71.34 | 84.51 |
| Succinate (F10) | 71.64 | 81.06 |
| Acetate (F11) | 70.16 | 84.51 |
| Perjeta | 70.62 | 84.33 |

### A. pH

TABLE 20
pH profile of pertuzumab formulations F09, F10 and F11 under stress
condition (40° C.)

| Formulation | | | |
|---|---|---|---|
| system/Days | F09 | F10 | F11 |
| 0 | 6.2 | 6.1 | 5.2 |
| 3 | 6.1 | 6.0 | 5.2 |
| 7 | 6.1 | 5.9 | 5.2 |
| 14 | 6.0 | 6.2 | 5.2 |

### Observation:

No change in pH was observed for all formulations till day 14.

### B. Protein Concentration:

TABLE 61
Protein concentration profile by OD 280 data of pertuzumab formulations
F9, F10 and F11 under stress condition (40° C.)

| Formulation | | | |
|---|---|---|---|
| system/Days | F09 | F10 | F11 |
| 0 | 30.7 | 29.6 | 28.6 |
| 3 | 31.6 | 29.9 | 29.2 |
| 7 | 35.9 | 30.0 | 29.4 |
| 14 | 37.1 | 30.3 | 29.4 |

### Observation:

No change in protein concentration was observed for F10 and F11 formulations till day 14, but unexpectedly concentration increased till day 14 for F09 formulation.

### C. Osmolality:

TABLE 72
Osmolality purity profile of pertuzumab formulations F9, F10 and F11
under stress condition (40° C.)

| Formulation | | | |
|---|---|---|---|
| system/Days | F09 | F10 | F11 |
| 0 | 170 | 163 | 157 |
| 3 | 183 | 163 | 155 |
| 7 | 261 | 163 | 157 |
| 14 | 289 | 162 | 156 |

### Observation:

No change in osmolality was observed for F10 and F11 formulations till day 14, but F09 unexpectedly showed increased in osmolality.

### D. SE HPLC

TABLE 83
SE- HPLC % monomer, % HMW and % LMW profile of Pertuzumab formulations
F9, F10 and F11 under stress condition (40° C.)

| | Formulation system | | | | | | | | |
|---|---|---|---|---|---|---|---|---|---|
| | F09 | | | F10 | | | F11 | | |
| | | % | | | % | | | % | |
| | % | monomer | % | % | monomer | % | % | monomer | % |
| Days | HMW | peak | LMW | HMW | peak | LMW | HMW | peak | LMW |
| Perjeta | 0.2 | 99.8 | 0.1 | 0.2 | 99.8 | 0.1 | 0.2 | 99.8 | 0.1 |
| 0 | 0.2 | 99.8 | <0.1 | 0.3 | 99.7 | <0.1 | 0.2 | 99.8 | <0.1 |
| 3 | 0.2 | 99.7 | 0.1 | 0.4 | 99.5 | 0.1 | 0.2 | 99.8 | 0.1 |
| 7 | 0.2 | 99.7 | 0.2 | 0.4 | 99.4 | 0.2 | 0.2 | 99.6 | 0.2 |
| 14 | 0.2 | 99.5 | 0.3 | 0.5 | 99.2 | 0.3 | 0.2 | 99.5 | 0.4 |

### Observation:

No change in purity was observed for all formulations till day 14, only slight increase in % LMW was seen for all the formulations, however, the same were within the permissible limits.

### E. CEX HPLC

TABLE 94
CEX- HPLC % Monomer, % Acidic and % Basic purity profile of Pertuzumab
formulations F09, F10 and F11 under stress condition (40° C.)

| | Formulation system | | | | | | | | |
|---|---|---|---|---|---|---|---|---|---|
| | F09 | | | F10 | | | F11 | | |
| | % | % Main | % | % | % Main | % | % | % Main | % |
| Days | Acidic | peak | Basic | Acidic | peak | Basic | Acidic | peak | Basic |
| Perjeta | 18.9 | 68.8 | 12.4 | 18.9 | 68.8 | 12.4 | 18.9 | 68.8 | 12.4 |
| 0 | 15.6 | 75.9 | 8.6 | 16.0 | 75.4 | 8.6 | 15.7 | 75.9 | 8.5 |
| 3 | 16.9 | 72.4 | 10.7 | 17.9 | 72.0 | 10.1 | 18.1 | 71.3 | 10.6 |
| 7 | 21.9 | 68.0 | 10.1 | 21.5 | 68.1 | 10.4 | 20.6 | 68.6 | 10.8 |
| 14 | 27.6 | 62.7 | 9.7 | 27.6 | 63.1 | 9.3 | 26.0 | 63.0 | 11.0 |

### Observation:

There was increase in % acidic peak and decrease in % main peak observed for all the formulations till day 14.

TABLE 25
CEX- HPLC rate of degradation for % acidic,
main peak and % basic peak in CEX HPLC

| | Formulation system | | | | | |
|---|---|---|---|---|---|---|
| | F09 | | | F10 | | |
| | % | % Main | % | % | % Main | % |
| Days | Acidic | peak | Basic | Acidic | peak | Basic |
| Perjeta | 18.9 | 68.8 | 12.4 | 18.9 | 68.8 | 12.4 |
| 0 | 0.0 | 0.0 | 0.0 | 0.0 | 0.0 | 0.0 |
| 3 | 1.3 | 3.5 | 2.1 | 1.9 | 3.4 | 1.5 |
| 7 | 6.3 | 7.9 | 1.5 | 5.5 | 7.3 | 1.8 |
| 14 | 12.0 | 13.2 | 1.1 | 11.6 | 12.3 | 0.7 |
| Rate of | 0.86 | 0.94 | 0.08 | 0.83 | 0.88 | 0.05 |
| degradation | | | | | | |
| per day | | | | | | |

### Observation:

During stress stability study, rate of degradation was observed lower in succinate buffer containing formulation than compare to histidine buffer containing formulation for acidic impurities and % main peak shows succinate buffer is showing better

### Example 2

### Preparation of the HER2 Antibody Pertuzumab Formulation

### Step 1: Preparation of Buffer Formulation

Based on the results of the above studies, surprisingly succinate was found to be more preferable and accordingly, its readily available and economical source succinic acid was used as a buffer along with other excipients as listed in Table 26 to provide buffer solution as follows:
1) Each of the excipients in the quantities as per Table 26 were weighed.
2) Each of the excipients were dissolved one by one into water for injection.
3) pH of the solution was in range of about 4.9-6.5.
4) Volume was adjusted up to 100 ml using water for injection and in process testing was carried out.
5) Aseptic filtration was carried out using 0.2-micron filter.

### Step 2: Preparation of Concentrated Form of Bulk Solution

1) Active drug substance equivalent to target concentration of about 30 mg/mL (+10%) was weighed.
2) Weighed quantity of active drug substance was dissolved in 50%-80% volume of buffer formulation by stirring at optimum speed of about 100 rpm-about 200 rpm.
3) Adjustment of the pH was carried out using HCl/NaOH solution to target pH in the range of 5-7.7.
4) In process testing was carried out and final dilution was adjusted based on the results to achieve target concentration of the active drug substance to about 30 mg/ml (±10%).
5) pH was adjusted again (if required) and final volume was made up to 100 ml using formulation buffer prepared in step 1.
   Step 3: Aseptic Filtration and Filing into Containers
1) Aseptic filtration was carried out using 0.2-micron PES or PVDF filter and filling into sterile containers (USP Type I glass).
2) Labeling was carried out as per the label format, release testing and storage as per their storage condition till further use.
3) Stable formulation of Pertuzumab is derived from the Example 1. Composition of final formulation is mentioned in Table 26. Manufacturing process is followed as per described above. Three batch of drug product are prepared to study stability at different conditions, real time for 36 months at 2-8° C., accelerated for 6 months at 30° C. and stress for 1 month at 40° C.

TABLE 26
Compositions of pertuzumab formulation

| Excipients | Quantity (mg/mL) |
|---|---|
| Succinic acid | 1.18 |
| Sucrose | 41.1 |
| Polysorbate 20 | 0.2 |
| pH adjustment using HCl/NaOH to pH 6.0 | |
| WFI | 1 mL |

### Example 3

### Preparation of the Formulation of HER2 Antibody Trastuzumab

Formulations of Trastuzumab were prepared as per Examples 1 compositions and following the method as per Example 1, except that the Pertuzumab was replaced with Trastuzumab in all the compositions formulated.

### Example 4

### Stability Study

Formulations of pertuzumab were prepared as per manufacturing process described in Example 2. Real time real temperature stability up to 12 months at 2-8° C., accelerated temperature stability study at 30° C. and stress condition stability study at 40° C. were performed. 6 months and 12 months testing were performed in both upright and inverted condition. Formulation composition is mentioned in Table 27.

TABLE 27
Compositions of pertuzumab formulation

| Excipients | Quantity (mg/mL) |
|---|---|
| Succinic acid | 1.18 |
| Sucrose | 41.1 |
| Polysorbate 20 | 0.2 |
| pH adjustment using HCl/NaOH to pH 6.0 | |
| WFI | 1 mL |

### A. Real Time Stability Study

All the quality attributes tested are meeting set acceptance criteria. no significant change in pH, appearance, protein concentration, purity by SE HPLC and CEX HPLC was observed shows that pertuzumab formulation is stable at 2-8° C. recommended long term storage condition. Results of the real time stability study are mentioned below in Tables 28-30.

TABLE 28
Real time real temperature stability up to 12 months at 2-8° C. (Batch 1)

| | Stability time point in months | | | | | | | | |
|---|---|---|---|---|---|---|---|---|---|
| | | | | | 6 M | 6 M | | 12 | 12 M |
| Parameters | Initial | 1 M | 2 M | 3 M | (Up) | (Inv) | 9 M | (Up) | (Inv) |
| Physical | C | C | C | C | C | C | C | C | C |
| Appearance | | | | | | | | | |
| pH | 6.0 | 6.1 | 6.1 | 5.9 | 6.0 | 6.0 | 6.0 | 6.1 | 6.0 |
| Protein | 30 | 30 | 29 | 31 | 30 | 30 | 31 | 30 | 30 |
| Concentration by | | | | | | | | | |
| OD_{280 nm} (mg/ml) | | | | | | | | | |
| Size Exclusion (SE) | 100 | 99 | 99 | 99 | 99 | 99 | 99 | 98 | 98 |
| HPLC | | | | | | | | | |
| Cation exchange | 76 | 76 | 77 | 75 | 75 | 75 | 74 | 74 | 74 |
| (CEX)HPLC | | | | | | | | | |
| Potency by in vitro | 97 | 97 | 96 | 94 | 97 | 103 | 121 | 94 | 99 |
| Bioassay | | | | | | | | | |

TABLE 29
Real time real temperature stability up to 12 months at 2-8° C. (Batch 2)

| | Stability time point in months | | | | | | | | |
|---|---|---|---|---|---|---|---|---|---|
| | | | | | 6 M | 6 M | | 12 | 12 M |
| Parameters | Initial | 1 M | 2 M | 3 M | (Up) | (Inv) | 9 M | (Up) | (Inv) |
| Physical | C | C | C | C | C | C | C | C | C |
| Appearance | | | | | | | | | |
| pH | 6.1 | 6.1 | 6.1 | 5.9 | 6.0 | 6.0 | 6.0 | 6.1 | 6.0 |
| Protein | 30 | 29 | 29 | 30 | 29 | 29 | 30 | 29 | 29 |
| Concentration by | | | | | | | | | |
| OD_{280 nm} (mg/ml) | | | | | | | | | |
| Size Exclusion (SE) | 100 | 99 | 99 | 99 | 99 | 99 | 99 | 97 | 97 |
| HPLC | | | | | | | | | |
| Cation exchange | 76 | 75 | 75 | 74 | 75 | 75 | 73 | 74 | 73 |
| (CEX)HPLC | | | | | | | | | |
| Potency by in vitro | 84 | 102 | 94 | 91 | 93 | 90 | 112 | 95 | 101 |
| Bioassay | | | | | | | | | |

TABLE 30
Real time real temperature stability up to 12 months at 2-8° C. (Batch 3)

| | Stability time point in months | | | | | | | | |
|---|---|---|---|---|---|---|---|---|---|
| | | | | | 6 M | 6 M | | 12 | 12 M |
| Parameters | Initial | 1 M | 2 M | 3 M | (Up) | (Inv) | 9 M | (Up) | (Inv) |
| Physical | C | C | C | C | C | C | C | C | C |
| Appearance | | | | | | | | | |
| pH | 6.1 | 6.1 | 6.1 | 5.9 | 6.0 | 6.0 | 6.1 | 6.0 | 6.1 |
| Protein | 29 | 28 | 29 | 29 | 28 | 28 | 30 | 29 | 29 |
| Concentration by | | | | | | | | | |
| OD_{280 nm} (mg/ml) | | | | | | | | | |
| Size Exclusion (SE) | 100 | 100 | 100 | 100 | 100 | 100 | 99 | 99 | 98 |
| HPLC | | | | | | | | | |
| Cation exchange | 75 | 74 | 75 | 74 | 73 | 75 | 72 | 72 | 73 |
| (CEX)HPLC | | | | | | | | | |
| Potency by in vitro | 96 | 89 | 104 | 97 | 98 | 101 | 119 | 99 | 101 |
| Bioassay | | | | | | | | | |

### B. Accelerated Stability Study

All the quality attributes tested were found to meet the set acceptance criteria. no significant change in pH, appearance, protein concentration, purity by SE HPLC and CEX HPLC was observed shows that pertuzumab formulation is stable at 30° C. However, decrease in % main peak in CEX HPLC was observed with increase in % acidic/basic impurities but it is within set acceptance criteria. Results of the accelerated stability study are mentioned below in Tables 31-33.

TABLE 31
Accelerated temperature stability up to 6 months at 30° C. (Batch 1)

| | Stability time point in months | | | | | | |
|---|---|---|---|---|---|---|---|
| | | | | 3 M | 3 M | 6 M | 6 M |
| Parameters | Initial | 1 M | 2 M | (Up) | (Inv) | (Up) | (Inv) |
| Physical Appearance | C | C | C | C | C | C | C |
| pH | 6.0 | 6.1 | 6.1 | 5.9 | 5.9 | 6.0 | 6.0 |
| Protein Concentration by | 30 | 30 | 30 | 31 | 30 | 29 | 30 |
| OD_{280 nm} (mg/ml) | | | | | | | |
| Size Exclusion (SE) HPLC | 100 | 99 | 99 | 98 | 98 | 98 | 98 |
| Cation exchange (CEX) HPLC | 76 | 70 | 68 | 63 | 63 | 53 | 53 |
| Potency by in vitro Bioassay | 97 | 91 | 81 | 95 | 84 | 92 | 92 |

TABLE 32
Accelerated temperature stability
up to 6 months at 30° C. (Batch 2)

| | Stability time point in months | | | | | | |
|---|---|---|---|---|---|---|---|
| | | | | 3 M | 3 M | 6 M | 6 M |
| Parameters | Initial | 1 M | 2 M | (Up) | (Inv) | (Up) | (Inv) |
| Physical | C | C | C | C | C | C | C |
| Appearance | | | | | | | |
| pH | 6.1 | 6.1 | 6.1 | 6.0 | 5.9 | 6.0 | 6.0 |
| Protein | 30 | 29 | 29 | 30 | 29 | 29 | 28 |
| Concentration by | | | | | | | |
| OD_{280 nm} (mg/ml) | | | | | | | |
| Size Exclusion | 100 | 99 | 98 | 98 | 98 | 98 | 98 |
| (SE) HPLC | | | | | | | |
| Cation exchange | 76 | 70 | 67 | 64 | 62 | 52 | 53 |
| (CEX)HPLC | | | | | | | |
| Potency by in vitro | 84 | 88 | 96 | 87 | 104 | 88 | 90 |
| Bioassay | | | | | | | |

TABLE 33
Accelerated temperature stability
up to 6 months at 30° C. (Batch 3)

| | Stability time point in months | | | | | | |
|---|---|---|---|---|---|---|---|
| | | | | 3 M | 3 M | 6 M | 6 M |
| Parameters | Initial | 1 M | 2 M | (Up) | (Inv) | (Up) | (Inv) |
| Physical | C | C | C | C | C | C | C |
| Appearance | | | | | | | |
| pH | 6.1 | 6.1 | 6.1 | 6.0 | 5.8 | 6.1 | 6.0 |
| Protein | 29 | 29 | 29 | 29 | 28 | 28 | 28 |
| Concentration by | | | | | | | |
| OD_{280 nm} (mg/ml) | | | | | | | |
| Size Exclusion | 100 | 99 | 99 | 98 | 98 | 99 | 99 |
| (SE) HPLC | | | | | | | |
| Cation exchange | 75 | 69 | 65 | 61 | 61 | 51 | 52 |
| (CEX)HPLC | | | | | | | |
| Potency by in | 96 | 85 | 93 | 92 | 100 | 91 | 95 |
| vitro Bioassay | | | | | | | |

### C. Stress Stability Study

One batch was use for stress stability study (batch 3). All the quality attributes tested are meeting set acceptance criteria. no significant change in pH, appearance, protein concentration, purity by SE HPLC was observed shows that pertuzumab formulation is stable at 40° C. for 1 month. However, decrease in % main peak in CEX HPLC was observed with increase in % acidic/basic impurities but it is within set acceptance criteria. Results of the stress stability study are mentioned below in table 33.

TABLE 34
Stress stability up to 1 months at 40° C. (Batch 3)

| | Stability time point in weeks | | | | |
|---|---|---|---|---|---|
| Parameters | Initial | 1 W | 2 W | 3 W | 4 W |
| Physical Appearance | C | C | C | C | C |
| pH | 6.1 | 6.1 | 6.1 | 6.1 | 6.1 |
| Protein Concentration (mg/ml) | 29 | 29 | 29 | 29 | 29 |
| Size Exclusion (SE) HPLC | 99 | 99 | 99 | 99 | 99 |
| Cation exchange (CEX) | 73 | 68 | 63 | 60 | 58 |
| HPLC | | | | | |
| Potency by in vitro Bioassay | 100 | 94 | 97 | 105 | 101 |

### Advantages of the Present Invention

The present invention provides pharmaceutical composition comprising HER2 antibody, formulations thereof and process for preparing the same that can satisfy the existing needs and/or can generally overcome one or more deficiencies found in the existing art.

The present invention provides liquid pharmaceutical composition comprising HER2 antibody, its formulation, and process for preparation of the same that is economically viable to produce and/or easily scalable.

The present invention provides a liquid formulation comprising HER2 antibody, wherein the liquid composition is stable at various temperatures and the present invention provides a process for preparing the such formulation.

The present invention provides a liquid formulation comprising HER2 antibody for example, pertuzumab and trastuzumab and process for preparing the same wherein the liquid formulation is stable at various temperatures.

We claim:

1. A pharmaceutical composition comprising:
a) a HER2 antibody selected from pertuzumab and trastuzumab, analogues or complexes thereof;
b) a buffer;
c) a stabilizer; and
d) a surfactant,
wherein the pH of the composition ranges from about 4.7 to about 6.5, preferably from about 4.9 to about 6.3.

2. The pharmaceutical composition as claimed in claim 1, wherein the buffer is selected from the group consisting of succinic acid, succinate, phosphate, acetate, citrate, tromethamine (Tris), sodium phosphate, citro phosphate, sodium phosphate dibasic dehydrate, tromethamine (Tris) acetate, tromethamine (Tris) phosphate, glutamic acid, glycine, histidine, histidine hydrochloride, arginine, or combinations thereof.

3. The pharmaceutical composition as claimed in claim 1, wherein the stabilizer is selected from the group consisting of sucrose, glycerol, propylene glycol, mannitol, sorbitol, PEG 400, trehalose or any combination thereof.

4. The pharmaceutical composition as claimed in claim 1, wherein the surfactant is selected from the group consisting of polysorbate 20, polysorbate 80, tween, polyethylene glycol, sodium dodecyl sulphate (SDS), poloxamer or any combinations thereof.

5. The pharmaceutical composition as claimed in claim 1, wherein the buffer is in an amount ranging from 3 mM to 100 mM, preferably from 3 mM to 60 mM.

6. The pharmaceutical composition as claimed in claim 1, wherein the stabilizer is present in the concentration ranging from 20 mg/ml to 100 mg/ml of the composition.

7. The pharmaceutical composition as claimed in claim 1, wherein the surfactant is present in an amount ranging from about 0.1 mg/ml to about 0.5 mg/ml w/v of the composition.

8. A pharmaceutical composition comprising:
a) a HER2 antibody selected from pertuzumab and trastuzumab;
b) a buffer being succinic acid;
c) a stabilizer being sucrose; and
d) a surfactant being Polysorbate 20.

9. A pharmaceutical formulation comprising:
a) a HER2 antibody selected from pertuzumab or trastuzumab, analogues or complexes thereof in an amount from about 10 mg/ml to about 100 mg/ml;
b) a buffer in an amount from about 5 mM to about 30 mM;
c) a stabilizer in an amount from 20 mg/ml to about 100 mg/ml; and
d) a surfactant in an amount from 0.1 mg/ml to about 0.5 mg/ml.

10. The pharmaceutical formulation as claimed in claim 9, wherein the formulation comprises:
e) the HER2 antibody selected from pertuzumab and trastuzumab;
f) the buffer being succinic acid;
g) the stabilizer being sucrose; and
h) the surfactant being Polysorbate 20.

11. The pharmaceutical formulation as claimed in claim 10, wherein the formulation is a liquid formulation for injection.

12. A process for preparing HER2 antibody formulation for injection, said process comprising:
a) dissolving a buffering agent, a stabilizer and a surfactant in water for injection to obtain a buffer solution;
b) dissolving a HER2 antibody into the buffer formulation solution of step (a) to obtain a bulk solution; and
c) adjusting pH of formulated bulk solution of step (b) with a pH-adjusting agent to a pH range from about 4.7 to about 6.5, preferably at 4.9 to 6.3.

13. The process as claimed in claim 12, wherein the buffering agent comprises succinic acid; the stabilizer comprises sucrose; the surfactant comprises Polysorbate 20.

14. The process as claimed in claim 13, wherein the pH-adjusting agent is selected from hydrochloric acid, sodium hydroxide with concentration ranging from 0.1N to 5N.
